(19) United States
(12) Patent Application Publication
(10) Pub. No.: US 2024/0269061 A1
(43) Pub. Date: Aug. 15, 2024

(54) Skin Care Composition

(71) Applicant: Mary King, Clearwater, FL (US)

(72) Inventor: Mary King, Clearwater, FL (US)

(21) Appl. No.: 18/110,218

(22) Filed: Feb. 15, 2023

(51) Int. Cl. A61K 8/9789; A61K 8/36; A61K 8/67; A61K 8/9794; A61Q 19/00

(52) U.S. Cl. CPC A61K 8/9789; A61K 8/36; A61K 8/676; A61K 8/9794; A61Q 19/007; A61K 2800/5922

(57) ABSTRACT

A skin care composition including purified water, Boswellia serrata, Hyssopus officinalis, Cucumis sativus juice, Aloe vera gel, Lavandula oil, apple cider vinegar, Origanum vulgare oil, Phyllanthus emblica, ascorbic acid, Cinnamomum verum.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to skin care solution mixture and more particularly pertains to a new skin care solution mixture for placement on a person's skin to keep the skin healthy.

### (2) Description of Related Art Including Information Disclosed Under 37 CFR 1.97 and 1.98

The prior art relates to skin care solution mixtures used to reduce inflation and to cure dry skin.

### (h) BRIEF SUMMARY OF THE INVENTION

An embodiment of the disclosure meets the needs presented above by generally comprising a mixture including purified water, Boswellia serrata, Hyssopus officinalis, Cucumis sativus juice, Aloe vera gel, Lavandula oil, apple cider vinegar, Origanum vulgare oil, Phyllanthus emblica, ascorbic acid, Cinnamomum verum.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

Not Applicable

### (j) DETAILED DESCRIPTION OF THE INVENTION

Now a new skin care solution mixture embodying the principles and concepts of an embodiment of the disclosure will be described.

The skin care composition generally comprises a mixture configured to be place on skin to condition the skin, said mixture comprising, generally, purified water, Boswellia serrata, Hyssopus officinalis, Cucumis sativus juice, Aloe vera gel, Lavandula oil, apple cider vinegar, Origanum vulgare oil, Phyllanthus emblica, ascorbic acid, and Cinnamomum verum.

In one embodiment, the mixture more specifically includes by volume:
at least 85% purified water;
between 0.02% and 0.1% Boswellia serrata;
between 0.04% and 0.12% Hyssopus officinalis;
between 2.0% and 3.0% Cucumis sativus juice;
between 4.0% and 5.0% Aloe vera gel;
between 0.01% and 0.06% Lavandula oil;
between 2.0% and 3.0% apple cider vinegar;
between 0.02% and 0.1% Origanum vulgare oil;
between 0.01% and 0.06% Phyllanthus emblica;
between 0.01% and 0.05% ascorbic acid; and between 0.005% and 0.03% Cinnamomum verum.

In another embodiment, the mixture includes by volume:
91% purified water;
0.06% Boswellia serrata;
0.07% Hyssopus officinalis;
2.26% Cucumis sativus juice;
4.25% Aloe vera gel;
0.03% Lavandula oil;
2.26% apple cider vinegar;
0.05% Origanum vulgare oil;
0.03% Phyllanthus emblica;
0.02% ascorbic acid; and
0.01% Cinnamomum verum.

Each of the above components are commonly available. However, it is noted that Bowellia serrata is typically known as bosweillia and is found in capsule form. The common name for Hyssopus officinalis and is commonly found in capsule form under the name Hyssop. Cucumis sativus is common cucumber juice. Lavandula is typically sold as lavender oil. The apple cider vinegar may comprise any common apple cider vinegar. Origanum vulgare is typically sold as oil of oregano. Phyllanthus emblica is commonly sold under the name Amla or Indian Gooseberry and can be found in capsule form. Ascorbic acid is generally known and sold as vitamin C. Cinnamomum verum is common cinnamon and is also sold in capsule form.

In one embodiment, a method of making the skin care composition includes the steps of:
1) adding to 2.4 gallons of water the bosweilla, hyssop, aloe vera gel, lavender oil, apple cider vinegar, oregano, Amla, vitamin C and cinnamon to water;
2) creating cucumber juice by cutting an unpeeled cucumber into small pieces and placing the pieces in approximately 8 ounces of water for 2 hours and then removing the cucumber pieces and seeds;
3) pouring the cucumber juice into the 2.4 gallons of water to define a solution; 4) letting the solution sit for 2 hours;
5) pour solution through a strainer and into containers for storage and can be stored at room temperature.

In use, the solution is shaken before use. After being shaken, the solution is placed on a person's skin to keep skin healthy and treat skin irritations including cuts, burns, acne, psoriasis, skin fungus, rashes, sores and the like.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A mixture configured to be placed on skin to condition the skin, said mixture comprising:
purified water;
Boswellia serrata;
Hyssopus officinalis;
Cucumis sativus juice;
Aloe vera gel;
Lavandula oil;
apple cider vinegar;
Origanum vulgare oil;
Phyllanthus emblica;
ascorbic acid; and
Cinnamomum verum.

2. A mixture configured to be placed on skin to condition the skin, said mixture comprising by volume:
at least 85% purified water;
between 0.02% and 0.1% Boswellia serrata;
between 0.04% and 0.12% Hyssopus officinalis;
between 2.0% and 3.0% Cucumis sativus juice;
between 4.0% and 5.0% Aloe vera gel;
between 0.01% and 0.06% Lavandula oil;
between 2.0% and 3.0% apple cider vinegar;
between 0.02% and 0.1% Origanum vulgare oil;
between 0.01% and 0.06% Phyllanthus emblica;
between 0.01% and 0.05% ascorbic acid; and
between 0.005% and 0.03% Cinnamomum verum.
